(19) United States
(12) Patent Application Publication
(10) Pub. No.: US 2004/0256745 A1
(43) Pub. Date: Dec. 23, 2004

(54) Oxygen inhalation apparatus and method

(76) Inventor: Dominic Simler, London (GB)

Correspondence Address: Schwegman, Lundberg, Woessner & Kluth, P.A., P.O. Box 2938, Minneapolis, MN, 55402, US

(21) Appl. No.: 10/817,773

(22) Filed: Apr. 2, 2004

(30) Foreign Application Priority Data
Apr. 4, 2003 (GB) 0307876.3

(51) Int. Cl. B01F 3/04; A61M 11/00; A23L 1/00; A23F 3/00

(52) U.S. Cl. 261/121.1; 128/200.14; 99/275; 261/DIG. 65; 261/123

(57) ABSTRACT

In order to provide a method or apparatus whereby oxygen can be inhaled to produce an enhanced feeling of euphoria, oxygen enriched air is fed through an alcoholic drink in a container 6 so that alcohol vapour and flavours enter the oxygen enriched air. This air comprising oxygen and alcohol vapour can be inhaled at 7.

### CROSS-REFERENCE TO RELATED APPLICATION

This application claims priority under 35 USC 119 to United Kingdom Application No. 0307876.3 filed Apr. 4, 2003, which applications and publications are herein incorporated by reference.

The present invention relates to an apparatus and method for inhalation of oxygen.

It has been discovered in the past that a number of benefits can be obtained by inhalation of pure oxygen or oxygen enriched air. It is found that oxygen enriched air can improve memory, reaction time, performance and recovery from exertion. Apparatus has become available for making oxygen enriched air available, typically for therapeutic uses, for example in hospitals or for use in exercise.

Sources of oxygen enriched air comprise cylinders containing compressed oxygen gas, air generators or oxygen concentrators which take atmospheric air and separate out the oxygen and the nitrogen, for example by fractional adsorption of the gases onto molecular sieve. The results can be a supply of oxygen enriched air of up to 95% by volume of oxygen.

Oxygen enriched air has also been used in therapy such as aromatherapy, mixed with flavouring or aromas. For example, US2002/0189608 discloses a method of enhancing athletic performance by the administration of peppermint odour. DE20114663 discloses a spray dosing apparatus which may include oxygen and herb extract. U.S. published application 2002/0189608 and DE20114663 are hereby incorporated by reference for any purpose.

The present inventor has discovered that a mixture of oxygen enriched air and the alcohol vapour from an alcoholic drink can be inhaled which induces a sense of well-being euphoria or relaxation.

Small amounts of alcohol may be employed for example in the disclosure of DE20114663 for solublising plant extracts. However, the quantities required will be very small. The present inventor has realised that if an alcoholic drink, particularly a spirit is used as a source of alcohol, a particularly strong effect can be obtained accompanied by appropriate flavours which are familiar to users.

Accordingly, the present invention provides oxygen inhalation apparatus, comprising:

a source of oxygen enriched air,

a container comprising an alcoholic drink,

means contacting the oxygen enriched air with the alcoholic drink so that alcohol vapour enters the oxygen enriched air and

means for allowing the oxygen enriched air and alcohol vapour to be inhaled.

The present invention further provides a method of oxygen inhalation, comprising:

providing a source of oxygen enriched air

providing a source of alcoholic drink,

contacting the oxygen enriched air with the alcoholic drink so that alcohol vapour enters the oxygen enriched air, and delivering the oxygen enriched air and alcohol vapour so that they can be inhaled.

Any suitable source of oxygen enriched air may be used. A source of oxygen may be used alone or the oxygen content of a supply of air can be raised by feeding to the supply of air a feed of oxygen rich gas. For example, compressed oxygen in a gas cylinder may be used. An air generator or oxygen generator may also be used. However, it is preferred to use an oxygen concentrator of the type known in the art which can separate the oxygen from atmospheric air to supply oxygen enriched air. A combination of these sources may be used. These sources may be combined with a supply of air to raise the oxygen content of the supply of air.

The oxygen enriched air comprises in excess of 21% by volume of oxygen. It is found that a greater effect can be obtained if the level of oxygen is higher. Preferably, the oxygen enriched air comprises 40% by volume, more preferably in excess of 60% by volume oxygen, most preferably in excess of 90% by volume of oxygen. Oxygen enriched air is suitably supplied at a rate of 3 litres per minute or more, preferably 5 litres per minute or more, most preferably 6 litres per minute.

The oxygen enriched air may comprise other gases, for example nitrogen gas, carbon dioxide, water vapour and flavouring elements from the alcoholic drink.

The container comprising the alcoholic drink and the means for contacting the oxygen enriched air with the alcoholic drink may be part of the same apparatus. For example, the means for contacting the oxygen enriched air with the alcoholic drink may comprise a conventional nebuliser of the type used in medical apparatus or a bubble jar in which the air is bubbled through the liquid alcoholic drink. Any other system for contacting the oxygen enriched air with the alcohol may be used, for example a spray or a container with surfaces moistened with the alcoholic drink.

A heater may be provided to enhance the transfer of alcohol vapour to the oxygen enriched air.

The means for allowing the oxygen enriched air to be inhaled may comprise any suitable means. For example, a mouthpiece may be provided.

A replicable mouthpiece, for example a plastic or cardboard tube can be used so that different users can use a single source of oxygen enriched air. Alternatively, a face mask may be provided for feeding the oxygen enriched air with alcohol vapour to the mouth and nose of the user. Alternatively, a nose hose may be provided for delivering alcohol enriched air and oxygen to the nostrils of the user.

The oxygen enriched air delivered by the method of apparatus of the present invention may also comprise water/alcohol droplets. This is particularly the case where a nebuliser is used.

All of this equipment is well known from medial applications.

In order to control the apparatus, means may be provided for controlling the rate of feed of oxygen enriched air. For example, a standard valve may be provided.

Means for measuring the flow may be provided, for example a rotameter or floating ball rate meter.

The use of an alcoholic drink to supply the alcohol vapour has many advantages.

In the first place, all commercially available alcoholic drinks are automatically suitable for human consumption. They are not commercially available until they have tested for acceptability.

Alcoholic drinks have familiar flavours which users enjoy and recognize.

Alcoholic drinks are widely available so that it is very easy to use the apparatus.

Any suitable alcoholic drink may be used. However, it is particularly preferred to use alcoholic drinks which have relatively high alcohol content, for example in excess of 20% by volume, more preferably in excess of 25% by volume, most preferably in excess of 30% by volume.

For example, spirits, cocktails, fortified wines and the like may be used.

It is particularly preferred to use alcoholic drinks which have additional flavour components, for example fruit flavour other types of flavour. It is found that the flavour is transferred effectively to the oxygen enriched air.

It is surprisingly found that the synergistic effect of inhaling oxygen and the alcohol vapour together allows the alcohol to be effective at a relatively small dose compared to drinking the alcoholic drink.

For example, whereas the normal single measure of spirits may be of the order of 25 milliliters, it is found that a greater effect can be obtained with the apparatus and the method of the invention even when the container contains only 5-10 milliliters of the alcoholic drink.

The alcohol content of oxygen enriched air may be in the range 0.01 grams per litre to 0.1 grams per litre.

The present invention will be described further below by way of example only with reference to the accompanying drawings in which

### BRIEF DESCRIPTION OF THE DRAWINGS

FIG. 1 is a schematic illustration of the apparatus for oxygen inhalation according to the invention.

FIG. 2 is a schematic illustration, at a larger scale, of a nebuliser, used with the present invention.

FIG. 3 is a schematic illustration of a different scale, of a bubble jar for use with the present invention.

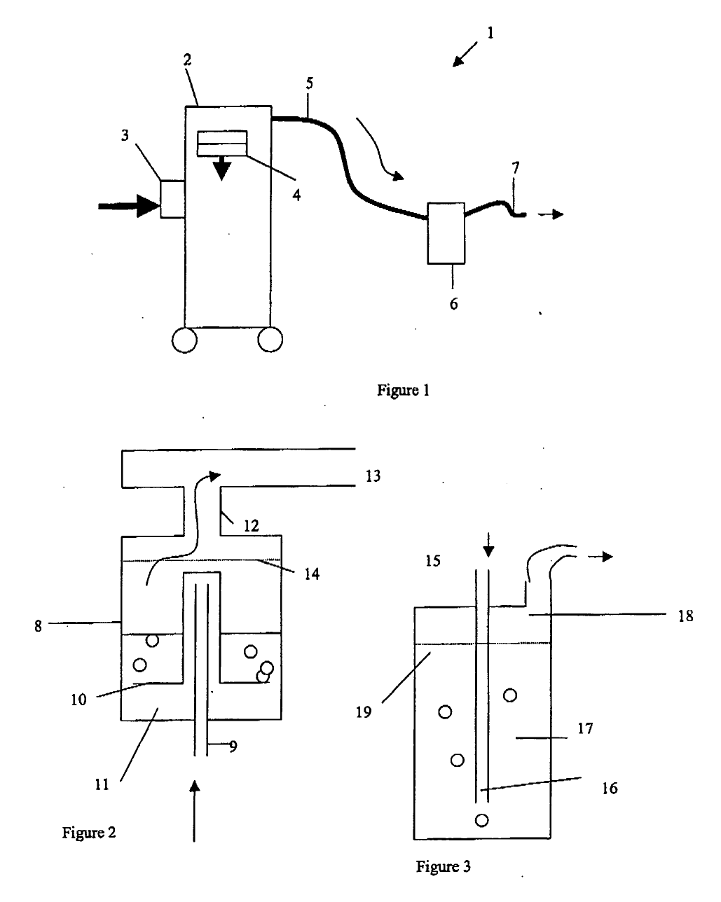

The oxygen inhalation apparatus 1 shown in FIG. 1 comprises an oxygen concentrator 2. This is a commercially available piece of equipment. Such equipment is typically of the size of a small traveling case. It is shown on wheels.

Atmospheric air is drawn in through a duct 3. Inside the oxygen contractor 2, which is supplied with electrical power by a suitable lead (not shown), fractional adsorption of oxygen nitrogen on to molecular sieve used to separate the oxygen and nitrogen. Nitrogen is exhausted through louvers 4. Oxygen enriched air comprising approximately 95% by volume of oxygen is delivered along a hose 5. The hose 5 is connected to a container for an alcoholic drink 6 where the oxygen enriched air is put into contact with the alcoholic drink so that alcohol vapour (and flavours from the alcoholic drink) enter the oxygen enriched air which is then delivered by a conduit 7, such as a mouthpiece, oxygen mask or inhaler to a user.

FIG. 2 shows a schematic view of a nebuliser, at an enlarged scale. Such nebulisers are currently available in the medical art and is shown here by way of example only.

Oxygen enriched air is delivered from a hose 5 to a tube 9 whence it is delivered under an annular disk through an alcoholic drink 11. In this way, the oxygen enriched air entrains alcohol vapour and, to some extent, droplets of alcoholic drink which are collected at the top of the nebuliser at 12 and delivered through a tube 13. A replaceable mouthpiece may be provided (not shown) for reasons of hygiene.

The nebuliser 8 typically comprises two parts which can be fixed together by screwing or by a push fit. By taking the top part off, a measured quantity of alcoholic drink can be placed into the lower part of the nebuliser 8.

It is found in practice that, where oxygen enriched air of at least 50% by volume, and preferably 90% by volume oxygen is used, at a flow rate in excess of 3 litres per minute, preferably in excess of 5 litres per minute, the measured dose of alcoholic drink need only be in the range 5-10 milliliters.

It is found in practice that, where oxygen enriched air of at least about 50% by volume, and preferably about 90% by volume oxygen is used, at a flow rate in excess of about 3 litres per minute, preferably in excess of about 5 litres per minute, the measured dose of alcoholic drink need only be in the range of about 5-10 milliliters. In an embodiment, air is enriched with at least 60% oxygen by volume. In an embodiment, air is enriched with at least 70% oxygen by volume. In an embodiment, air is enriched with at least 80% oxygen by volume. In an embodiment, the measured dose of alcoholic drink is about 6 milliliters. In an embodiment, the measured dose of alcoholic drink is about 7 milliliters. In an embodiment, the measured dose of alcoholic drink is about 8 milliliters. In an embodiment, the measured dose of alcoholic drink is about 9 milliliters.

FIG. 3 shows a bubble jar for putting the alcoholic drink into contact with the oxygen enriched air. Oxygen enriched air is delivered along the tube 16 so that it is bubbled through the alcoholic drink 17 and collected at 18 for connection to a further hose. The bubble jar comprises a top half and a bottom half secured together at 19 by a screw fit or push fit. The alcoholic drink can be placed in the bubble jar.

A flow meter, for example a rotameter or floating ball through a meter can be inserted in the hose 5 or conduit 7 or in a part of the nebuliser 8 or bubble jar 15 to measure the quantity of oxygen enriched air being delivered.

The present invention has been described above purely by way of example only and modifications can be made within the spirit of the invention, which extends to equivalents of the features described. The invention also consists in any individual features described or implicit herein or shown or implicit in the drawings or any combination of any such features or any generalization of any such features or combinations.

1. An oxygen inhalation apparatus comprising:
a source of oxygen enriched air
a container compromising alcoholic drink,
a contactor for contacting the oxygen enriched air with the alcoholic drink so that alcohol vapour enters the oxygen enriched air, and
an inhaler for allowing the oxygen enriched air and alcohol vapour to be inhaled.

2. The oxygen inhalation apparatus according to claim 1, wherein the rate of flow of oxygen enriched air is in excess of 3 litres per minute.

3. The oxygen inhalation apparatus according to claim 1, wherein the rate of flow of oxygen enriched air is in excess of 5 litres per minute.

4. The oxygen inhalation apparatus according to claim 1, wherein the rate of flow of oxygen enriched air is in excess of 6 litres per minute.

5. The oxygen inhalation apparatus according to claim 1, wherein the source of oxygen enriched air provides air comprising at least 50% by volume of oxygen.

6. The oxygen inhalation apparatus according to claim 1, wherein the source of oxygen enriched air provides air comprising at least 90% by volume of oxygen.

7. The oxygen inhalation apparatus according to claim 1, wherein the alcoholic drink comprises at least 20% by volume of alcohol.

8. The oxygen inhalation apparatus according to claim 1, wherein the alcoholic drink comprises at least 30% by volume of alcohol.

9. The oxygen inhalation apparatus according to claim 1, wherein the alcoholic drink is flavoured.

10. The oxygen inhalation apparatus according to claim 1, wherein the container is adapted to provide a measured dose in a range of about 5-10 milliliters.

11. A method of oxygen inhalation comprising:
providing a source of oxygen enriched air,
contacting the oxygen enriched air with an alcoholic drink so that alcohol vapour enters the oxygen enriched air, and
delivering the oxygen enriched air and alcohol vapour to an inhaler for allowing the oxygen enriched air to be inhaled.

12. The method of claim 11, wherein providing a source of oxygen enriched air includes flowing the oxygen enriched air at a rate of about 3 to 6 liters per minute.

13. The method of claim 12, wherein providing a source of oxygen enriched air includes providing air with at least about 50% by volume of oxygen.

14. The method of claim 11, wherein providing a source of oxygen enriched air includes providing air with at least about 90% by volume of oxygen.

15. The method of claim 11, wherein contacting including contacting the oxygen enriched air with an alcoholic drink that is at least about 20% by volume alcohol.

16. The method of claim 11, wherein contacting including contacting the oxygen enriched air with an alcoholic drink that is at least about 30% by volume alcohol.

17. The method of claim 11, wherein the alcohol vapour includes a flavoring.
